(19) United States
(12) Patent Application Publication
(10) Pub. No.: US 2005/0281782 A1
(43) Pub. Date: Dec. 22, 2005

(54) Novel recombinant poxvirus composition and uses thereof

(76) Inventors: Howard Kaufman, New York, NY (US); Kenneth Flanagan, New York, NY (US)

Correspondence Address: LESLIE GLADSTONE RESTAINO;BROWN RAYSMAN MILLSTEIN & STEINER LLP, 163 MADISON AVENUE, PO BOX 1989, MORRISTOWN, NJ, 07962-1989, US

(21) Appl. No.: 10/873,032

(22) Filed: Jun. 21, 2004

(51) Int. Cl. A61K 48/00; C12N 15/863

(52) U.S. Cl. 424/93.2; 435/456; 435/235.1

(57) ABSTRACT

The present invention provides a recombinant pox virus composition comprising a nucleic acid sequence encoding chemokines as costimulatory molecules. The present invention further provides a host cell, a host animal, and a pharmaceutical composition comprising the recombinant pox virus composition. Also provided is a method for treating or preventing a neoplasm or infectious disease in a subject, using the pox virus composition and/or an SLC agent. Additionally, the present invention provides a method for promoting the proliferation of a CD4 T cell, comprising administering to the cell an SLC agent in an amount effective to directly promote the proliferation of the cell. Finally, the present invention provides a method for treating or preventing a neoplasm or infectious disease in a subject using cultured CD4 T cells.

### STATEMENT OF GOVERNMENT INTEREST

This invention was made with government support under NIH Grant No. K08 CA 79881. As such, the United States government has certain rights in this invention.

### BACKGROUND OF THE INVENTION

Chemokines are proteins which comprise the largest family of known cytokines. Originally, they were characterized by their ability to induce directional migration of immune cells to sites of infection, inflammation, and tumor growth, and activation of leukocytes. Chemokines are produced by a variety of cell types in response to various stimulations, such as antigens, pathogens, and other cytokines, which in turn bind and activate a number of the seven-transmembrane G protein-coupled receptor superfamily cell surface receptors. Studies have revealed that chemokines and their receptors play a pivotal role in host defense against microorganisms (e.g., HIV) and neoplasms.

Over 50 chemokines have been identified to date. These are categorized into four families (C, CC, CXC, and CX₃C) based on the pattern of cysteine residues near the amino terminus. CC chemokine family is the largest of the four families, comprising chemokines such as secondary lymphoid chemokine (SLC), EBV-induced molecule 1 ligand chemokine (ELC), macrophage inflammatory protein (MIP)-1α, MIP-1β, regulated upon activation normal T cell expressed and secreted (RANTES), etc. The CXC chemokine family also includes a large number of chemokines, such interferon inducible protein 10 (IP-10) and monokine induced by gamma interferon (MiG). C chemokine family only has two members, Lymphotactin α and β, while CX₃C chemokine family contains only one known member, fractalkine.

While the immune system is adept at recognizing and neutralizing the effects of various pathogens (e.g., bacteria, viruses, fungi, protozoa, and metazoa) and mutated self-cells (e.g., pre-cancer and cancer cells), failure of the immune system to perform its functions often results in disease (e.g., infection and cancer). Vaccines for neoplasm and infectious diseases represent a major field of current research. Vaccines are increasingly being used to enhance immune responses, and may be useful in augmenting responses against weak immuno-targets, such as hard to treat viruses or neoplasia. Neoplasia is a disease characterized by an abnormal proliferation of cells known as a neoplasm. Neoplasms may manifest in the form of a leukemia or a solid tumor, and may be benign or malignant. Cytokines, chemokines, and other costimulatory molecules have been co-introduced with antigens or pathogens to further boost host immune response. For example, U.S. Pat. No. 6,265,189 (the '189 patent), Pox virus containing DNA encoding a cytokine and/or a tumor associated antigen, discloses and claims a recombinant pox virus containing exogenous DNA coding for a cytokine, a tumor-associated antigen, or a cytokine and a tumor-associated antigen in a non-essential region of the pox virus genome. The '189 patent further discloses recombinant vaccinia virus containing multiple cytokines; for example, murine or human IL-2 plus IFNγ are cloned into recombinant vaccinia virus NYVAC (see Examples 22 and 23, respectively). The '189 patent also discloses methods of making and using such composition against a variety of pathogens and in immunotherapy.

The introduction of chemokine genes into neoplastic cells has been used to increase local production of these immune modulators, for the purpose of enhancing tumor immunogenicity and consequent host recognition and elimination of tumor (Dranoff et al., Vaccination with irradiated tumor cells engineered to secrete murine granulocyte-macrophage colony-stimulating factor stimulates potent, specific, and long-lasting anti-tumor immunity. Proc. Natl. Acad. Sci. USA 90:3539-43, 1993; Gansbacher et al., Retroviral gene transfer induced constitutive expression of interleukin-2 or interferon-gamma in irradiated human melanoma cells. Blood 80:2817-25, 1992).

To date, various methods of gene transformation have been examined, and pox viruses, especially vaccinia virus, have shown potential to be effective, efficient, low risk gene transfer vectors. Vaccinia virus has been extensively studied as a recombinant vaccine for cancer and possesses powerful adjuvant activity for generating both humoral and cellular immune responses. Vaccinia virus is a model vector for gene expression given the ease of construction, stability and reliability of recombinant vaccinia vectors (Moss, B., Vaccinia virus: a tool for research and vaccine development. Science 252:1662-7, 1991). Transgene expression in vaccinia virus results in translation and secretion of high levels of recombinant protein over a period of several days (Moss, B., Genetically engineered pox viruses for recombinant gene expression, vaccination, and safety. Proc. Natl. Acad. Sci. USA 93:11341-8, 1996). In addition, there is an extensive clinical experience with vaccinia virus in cancer patients documenting the safety and immunogenicity of recombinant pox viruses expressing tumor associated antigens and other immune modulatory genes (Moss, 1991, supra; Moss, B., Pox virus vectors: cytoplasmic expression of transferred genes. Curr. Opin. Genet. Dev. 3:86-90, 1993; Kaufman, et al., Insertion of interleukin-2 (IL-2) and interleukin-12 (IL-12) genes into vaccinia virus results in effective anti-tumor responses without toxicity. Vaccine 20:1862-9, 2002; McAneny, et al., Results of a phase I trial of a recombinant vaccinia virus that expresses carcinoembryonic antigen in patients with advanced colorectal cancer. Ann. Surg. Oncol. 3:495-500, 1996). Furthermore, vaccinia virus provides a potent danger signal for T cell immunity and may also serve as a dendritic cell and T cell maturation factor enhancing the ability to generate tumor-specific immunity (Matzinger, P., An innate sense of danger. Ann. N. Y. Acad. Sci. 961:341, 2002).

Despite the various methods for treating cancers and infectious diseases (such as AIDS), these diseases remain prevalent in all segments of society, and are often fatal. Accordingly, there remains a need in the art for compositions and methods for the prevention and treatment of a wide spectrum of infectious diseases and neoplasia.

### SUMMARY OF THE INVENTION

The present invention provides a recombinant vaccinia virus composition comprising a nucleic acid sequence selected from the group consisting of: a nucleic acid sequence encoding chemokines IP-10 and ELC; a nucleic acid sequence encoding chemokines IP-10, ELC, and RANTES; a nucleic acid sequence encoding chemokines IP-10, ELC, and MIP-1α; a nucleic acid sequence encoding chemokines IP-10, ELC, and MIP-1β; a nucleic acid sequence encoding chemokines IP-10, ELC, RANTES, and MIP-1α; a nucleic acid sequence encoding chemokines IP-10, ELC, RANTES, and MIP-1β; a nucleic acid sequence encoding chemokines IP-10, ELC, MIP-1α, and MIP-1β; a nucleic acid sequence encoding chemokines IP-10, ELC, RANTES, MIP-1α, and MIP-1β; a nucleic acid sequence encoding chemokines IP-10 and SLC; a nucleic acid sequence encoding chemokines IP-10, SLC, and RANTES; a nucleic acid sequence encoding chemokines IP-10, SLC, and MIP-1α; a nucleic acid sequence encoding chemokines IP-10, SLC, and MIP-1β; a nucleic acid sequence encoding chemokines IP-10, SLC, RANTES, and MIP-1α; a nucleic acid sequence encoding chemokines IP-10, SLC, RANTES, and MIP-1β; a nucleic acid sequence encoding chemokines IP-10, SLC, MIP-1α, and MIP-1β; a nucleic acid sequence encoding chemokines IP-10, SLC, RANTES, MIP-1α, and MIP-1β; a nucleic acid sequence encoding chemokines IP-10, SLC, and ELC; a nucleic acid sequence encoding chemokines IP-10, SLC, ELC, and RANTES; a nucleic acid sequence encoding chemokines IP-10, SLC, ELC, and MIP-1α; a nucleic acid sequence encoding chemokines IP-10, SLC, ELC, and MIP-1β; a nucleic acid sequence encoding chemokines IP-10, SLC, ELC, RANTES, and MIP-1α; a nucleic acid sequence encoding chemokines IP-10, SLC, ELC, RANTES, and MIP-1β; a nucleic acid sequence encoding chemokines IP-10, SLC, ELC, MIP-1α, and MIP-1β; and a nucleic acid sequence encoding chemokines IP-10, SLC, ELC, RANTES, MIP-1α, and MIP-1β. The present invention also provides a composition further comprising the recombinant vaccinia virus composition disclosed herein together with at least one nucleic acid sequence encoding at least one costimulatory factor. The present invention further provides a host cell, a host animal, and a pharmaceutical composition comprising the recombinant vaccinia virus composition.

Additionally, the present invention provides a method for treating or preventing a neoplasm or infectious disease in a subject, comprising administering to the subject a pharmaceutical composition comprising a recombinant vaccinia virus, wherein the virus comprises a nucleic acid sequence selected from the group consisting of: a nucleic acid sequence encoding chemokines IP-10 and ELC; a nucleic acid sequence encoding chemokines IP-10, ELC, and RANTES; a nucleic acid sequence encoding chemokines IP-10, ELC, and MIP-1α; a nucleic acid sequence encoding chemokines IP-10, ELC, and MIP-1β; a nucleic acid sequence encoding chemokines IP-10, ELC, RANTES, and MIP-1α; a nucleic acid sequence encoding chemokines IP-10, ELC, RANTES, and MIP-1β; a nucleic acid sequence encoding chemokines IP-10, ELC, MIP-1α, and MIP-1β; a nucleic acid sequence encoding chemokines IP-10, ELC, RANTES, MIP-1α, and MIP-1β; a nucleic acid sequence encoding chemokines IP-10 and SLC; a nucleic acid sequence encoding chemokines IP-10, SLC, and RANTES; a nucleic acid sequence encoding chemokines IP-10, SLC, and MIP-1α; a nucleic acid sequence encoding chemokines IP-10, SLC, and MIP-1β; a nucleic acid sequence encoding chemokines IP-10, SLC, RANTES, and MIP-1α; a nucleic acid sequence encoding chemokines IP-10, SLC, RANTES, and MIP-1β; a nucleic acid sequence encoding chemokines IP-10, SLC, MIP-1α, and MIP-1β; a nucleic acid sequence encoding chemokines IP-10, SLC, RANTES, MIP-1α, and MIP-1β; a nucleic acid sequence encoding chemokines IP-10, SLC, and ELC; a nucleic acid sequence encoding chemokines IP-10, SLC, ELC, and RANTES; a nucleic acid sequence encoding chemokines IP-10, SLC, ELC, and MIP-1α; a nucleic acid sequence encoding chemokines IP-10, SLC, ELC, and MIP-1β; a nucleic acid sequence encoding chemokines IP-10, SLC, ELC, RANTES, and MIP-1α; a nucleic acid sequence encoding chemokines IP-10, SLC, ELC, RANTES, and MIP-1β; a nucleic acid sequence encoding chemokines IP-10, SLC, ELC, MIP-1α, and MIP-1β; and a nucleic acid sequence encoding chemokines IP-10, SLC, ELC, RANTES, MIP-1α, and MIP-1β. The pharmaceutical composition may further comprise at least one nucleic acid sequence encoding at least one costimulatory factor and/or a pharmaceutically acceptable carrier.

The present invention also provides a method for treating or preventing a neoplasm or infectious disease in a subject, comprising administering to the subject a pharmaceutical composition comprising an SLC agent in an amount effective to promote the proliferation of CD4 T cells directly. In one embodiment, the method further comprises administering to the cell a costimulatory factor. In another embodiment, the pharmaceutical composition further comprises at least one anti-neoplasm or anti-infection agent and/or a pharmaceutically acceptable carrier.

In one aspect, the present invention provides a method for promoting the proliferation of a CD4 T cell, comprising administering to the cell an SLC agent in an amount effective to promote the proliferation of the cell directly. In one embodiment, the method further comprises administering to the cell a costimulatory factor.

The present invention also provides a method for treating or preventing a neoplasm or infectious disease in a subject, comprising the steps of: obtaining or generating a culture of T cells; optionally, contacting the T cells with an amount of T cell activation agent effective to activate the T cells; contacting the T cells with an SLC agent effective to promote CD4 T cell proliferation directly; and introducing the proliferated T cells into the subject in an amount effective to treat the neoplasm or infectious disease. In one embodiment, the method further comprises administering to the subject a costimulatory factor.

Additional aspects of the present invention will be apparent in view of the description which follows.

### BRIEF DESCRIPTION OF THE FIGURES

FIG. 1 depicts the construction and characterization of rVmSLC. BSC-1 cells were infected with either rVmSLC or rVLacZ (MOI=10) and incubated at 37° C. for 48 hours before cells and supernatants were collected. (A) Western blot analysis of cell lysates from infections with either rVLacZ (lane 1, 10 μl and lane 2, 20 μl) or rVmSLC (lane 3, 10 μl of lysate and lane 4, 20 μl of lysate). Recombinant mSLC protein was used as a positive control (lane 5, 0.5 μg and lane 6, 1 μg). (B) Supernatants or mSLC protein (rSLC), with or without anti-mSLC antibody pre-treatment, from the infected cells was used in a microchemotaxis assay. Chemotaxis of enriched T cells by supernatants from cells infected with rVmSLC was measured by transmigration across a 5 μm pore size membrane. Data represents the number of cells counted per hemacytometer field by Trypan blue exclusion. **, P<0.0001 vs. rVLacZ supernatant. (C) After a three hour incubation, samples were collected and stained for CD4 (dark bars), CD8 (open bars) or CD11c (hatched bars), and the migration index was determined as described in examples. Results are representative of three individual experiments.

FIG. 2 illustrates that SLC expression by vaccinia virus enhances anti-vaccinia T-cell responses only at lower doses of vaccine. Mice were injected i.p. with rVLacZ (▪) or rVmSLC (▴) at doses of 10⁴, 10⁵, 10⁶, or 10⁷ pfu, and CTL activity against vaccinia-infected targets was measured in a 4 hour ⁵¹Cr-release assay using vaccinia-infected CT26-CEA targets. Data are shown for an E:T ration of 80:1 and represents one of three independent experiments. *, P<0.05.

FIG. 3 shows that rVmSLC treatment enhances the infiltration of T cells within established tumors. 5-day established CT26-CEA tumors were injected with 10⁷ pfu of either rVmSLC (A) or rVLacZ (B). Tumors were collected five days later, fixed, and cut into 5 μm sections. Sections were stained for infiltrating T cells using a mAb against CD3. Selected area shown at 40× magnification. Isotype-matched controls demonstrated no staining (not shown).

FIG. 4 demonstrates that rVmSLC enhances the infiltration of CD4 T cells into established tumors. 5-day established CT26-CEA tumors were injected with 10⁷ pfu of either rVLacZ (□) or rVmSLC (∇). 2, 5 and 7 days later, tumors were removed, digested and quantitated by flow cytometry using mAbs to CD4 (A), CD8 (B) or CD11c (C). *, P<0.05.

FIG. 5 sets forth the effects of rVmSLC treatment on tumor growth. 5-day established CT26-CEA tumors were injected with either PBS (◯), 10⁷ pfu rVLacZ (□) or 10⁷ pfu rVmSLC (∇). Tumor growth was measured every 1-3 days by measuring the longest perpendicular diameters and data is presented as tumor area (mm²). **, P<0.01, ***P<0.001 compared to rVLacZ treated tumors (A). In a separate experiment, tumors treated with rVLacZ (□) or rVmSLC (∇) were collected at the indicated time points after vaccine administration and weighed (B). *, P<0.05 and **, P<0.005. Mice treated with rVmSLC also show improved survival (C).

FIG. 6 shows that rVmSLC mediates tumor regression through T cells. 5-day established CT26-CEA tumors were injected with 10⁷ pfu of either rVLacZ (▪) or rVmSLC (▴) after in vivo depletion of CD4 T cells (A), CD8 T (B) cells or both subsets of T cells (C) as described in the examples herein and tumor growth was measured as described and compared to rVmSLC treated immune-competent mice (∇). *, P<0.05, **, P<0.0 and ***, P<0.001.

FIG. 7 demonstrates that SLC induces proliferation of T cells. 2×10⁵ red blood cell (RBC)-depleted splenocytes (A) or enriched T cells (B) were cultured in the presence of increasing concentrations of SLC protein and proliferation was measured by standard ³H-Thymidine incorporation. *, P<0.05 and **, P<0.01.

### DETAILED DESCRIPTION OF THE INVENTION

The present invention relates to methods for treating and preventing infectious diseases and neoplasia. The present invention further provides compositions for treating and preventing infectious diseases and neoplasia and methods of making and using the same.

As used herein, the term “infectious disease” denotes a disease resulting from the presence and activity of a microbial agent, such as a prion, bacterium, fungus, protozoon, and virus as well as the toxins, pathogens, etc. generated as a result of its activity. “Neoplasia” refers to the uncontrolled and progressive multiplication of tumor cells, under conditions that would not elicit, or would cause cessation of, multiplication of normal cells. Neoplasia results in a “neoplasm”, which is defined herein to mean any new and abnormal growth, particularly a new growth of tissue, in which the growth of cells is uncontrolled and progressive. Thus, neoplasia includes “cancer”, which herein refers to a proliferation of tumor cells having the unique trait of loss of normal controls, resulting in unregulated growth, lack of differentiation, local tissue invasion, and/or metastasis.

As used herein, neoplasms include, without limitation, morphological irregularities in cells in tissue of a subject or host, as well as pathologic proliferation of cells in tissue of a subject, as compared with normal proliferation in the same type of tissue. Additionally, neoplasms include benign tumors and malignant tumors (e.g., colon tumors) that are either invasive or noninvasive. Malignant neoplasms are distinguished from benign neoplasms in that the former show a greater degree of anaplasia, or loss of differentiation and orientation of cells, and have the properties of invasion and metastasis. Examples of neoplasms or neoplasias from which the target cell of the present invention may be derived include, without limitation, carcinomas (e.g., squamous-cell carcinomas, adenocarcinomas, hepatocellular carcinomas, and renal cell carcinomas), particularly those of the bladder, bowel, breast, cervix, colon, esophagus, head, kidney, liver, lung, neck, ovary, pancreas, prostate, and stomach; leukemias; benign and malignant lymphomas, particularly Burkitt's lymphoma and Non-Hodgkin's lymphoma; benign and malignant melanomas; myeloproliferative diseases; sarcomas, particularly Ewing's sarcoma, hemangiosarcoma, Kaposi's sarcoma, liposarcoma, myosarcomas, peripheral neuroepithelioma, and synovial sarcoma; tumors of the central nervous system (e.g., gliomas, astrocytomas, oligodendrogliomas, ependymomas, gliobastomas, neuroblastomas, ganglioneuromas, gangliogliomas, medulloblastomas, pineal cell tumors, meningiomas, meningeal sarcomas, neurofibromas, and Schwannomas); germ-line tumors (e.g., bowel cancer, breast cancer, prostate cancer, cervical cancer, uterine cancer, lung cancer, ovarian cancer, testicular cancer, thyroid cancer, astrocytoma, esophageal cancer, pancreatic cancer, stomach cancer, liver cancer, colon cancer, and melanoma); mixed types of neoplasias, particularly carcinosarcoma and Hodgkin's disease; and tumors of mixed origin, such as Wilms' tumor and teratocarcinomas (Beers and Berkow (eds.), The Merck Manual of Diagnosis and Therapy, 17^{th} ed. (Whitehouse Station, N.J.: Merck Research Laboratories, 1999) 973-74, 976, 986, 988, 991). In one embodiment, the compositions and methods of the present invention may be utilized to prevent or treat a solid tumor.

In one aspect, the inventors provide a recombinant virus vector which comprises a nucleic acid sequence encoding at least one costimulatory factor. The costimulatory factor as used herein includes any molecules which are capable of enhancing immune responses to an antigen/pathogen in vivo and/or in vitro. It also includes any molecules which promote the activation, proliferation, differentiation, maturation, or maintenance of lymphocytes and/or other cells whose function is important or essential for immune responses. In one embodiment, the costimulatory factor may include chemokines (e.g., SLC, ELC, MIP-1α, MIP-1β, RANTES, IP-10, and MiG), cytokines (e.g., hematopoietin family of cytokines, such as IL2-13, GM-CSF; interferon family of cytokines, such as IFN α, β, γ; immunoglobulin superfamily of cytokines, such as B7.1, B7.2; TNF family of cytokines, such as TNF α and β, FasL, CD30L, CD40L, and 4-1BBL; as well as IL1, 16-18, and TGF β), the modulators of chemokines and cytokines expression and/or function, antigens, pathogens, and other immune modulators. As used herein, the costimulatory factor may be a polypeptide, nucleic acid, polysaccharide, lipid, small molecule compound, and fragments, variants, derivatives, and combinations thereof.

Unless otherwise indicated, “polypeptide” shall include a protein, protein domain, polypeptide, or peptide, and any fragment or variant or derivative thereof having polypeptide function. The variants preferably have greater than about 75% homology with the naturally-occurring polypeptide sequence, more preferably have greater than about 80% homology, even more preferably have greater than about 85% homology, and, most preferably, have greater than about 90% homology with the polypeptide sequence. In some embodiments, the homology may be as high as about 95%, 98%, or 99%. These variants may be substitutional, insertional, or deletional variants. The variants may also be chemically-modified derivatives: polypeptides which have been subjected to chemical modification, but which retain the biological characteristics of the naturally-occurring polypeptide. In one embodiment of the present invention, the polypeptide is mutated such that it has a longer half-life in vivo.

As used herein, a “nucleic acid” or “polynucleotide” includes a nucleic acid, an oligonucleotide, a nucleotide, a polynucleotide, and any fragment or variant thereof. The nucleic acid or polynucleotide may be double-stranded, single-stranded, or triple-stranded DNA or RNA (including cDNA), or a DNA-RNA hybrid of genetic or synthetic origin, wherein the nucleic acid contains any combination of deoxyribonucleotides and ribonucleotides and any combination of bases, including, but not limited to, adenine, thymine, cytosine, guanine, uracil, inosine, and xanthine hypoxanthine. The nucleic acid or polynucleotide may be combined with a carbohydrate, a lipid, a protein, or other materials. Preferably, the nucleic acid encodes costimulatory protein or nucleic acid (e.g., antisense RNA and small interference RNA (siRNA)).

The “complement” of a nucleic acid refers, herein, to a nucleic acid molecule which is completely complementary to another nucleic acid, or which will hybridize to the other nucleic acid under conditions of high stringency. High-stringency conditions are known in the art (see, e.g., Maniatis et al., Molecular Cloning: A Laboratory Manual, 2nd ed. (Cold Spring Harbor: Cold Spring Harbor Laboratory, 1989) and Ausubel et al., eds., Current Protocols in Molecular Biology (New York, N.Y.: John Wiley & Sons, Inc., 2001)). Stringent conditions are sequence-dependent, and may vary depending upon the circumstances. As used herein, the term “cDNA” refers to an isolated DNA polynucleotide or nucleic acid molecule, or any fragment, derivative, or complement thereof. It may be double-stranded, single-stranded, or triple-stranded, it may have originated recombinantly or synthetically, and it may represent coding and/or noncoding 5′ and/or 3′ sequences.

In one embodiment, the recombinant virus is a recombinant pox virus. Preferably, the recombinant pox virus is a vaccinia virus. More preferably, the recombinant pox virus is the WR strain of vaccinia virus.

In one embodiment, the recombinant virus vector comprises a nucleic acid sequence encoding at least one chemokine. In a preferred embodiment, the nucleic acid is selected from the group consisting of: a nucleic acid sequence encoding cytokines IP-10 and ELC; a nucleic acid sequence encoding cytokines IP-10, ELC, and RANTES; a nucleic acid sequence encoding cytokines IP-10, ELC, and MIP-1αc; a nucleic acid sequence encoding cytokines IP-10, ELC, and MIP-1β; a nucleic acid sequence encoding cytokines IP-10, ELC, RANTES, and MIP-1α; a nucleic acid sequence encoding cytokines IP-10, ELC, RANTES, and MIP-1β; a nucleic acid sequence encoding cytokines IP-10, ELC, MIP-1α, and MIP-1β; a nucleic acid sequence encoding cytokines IP-10, ELC, RANTES, MIP-1α, and MIP-1β; a nucleic acid sequence encoding cytokines IP-10 and SLC; a nucleic acid sequence encoding cytokines IP-10, SLC, and RANTES; a nucleic acid sequence encoding cytokines IP-10, SLC, and MIP-1α; a nucleic acid sequence encoding cytokines IP-10, SLC, and MIP-1β; a nucleic acid sequence encoding cytokines IP-10, SLC, RANTES, and MIP-1α; a nucleic acid sequence encoding cytokines IP-10, SLC, RANTES, and MIP-1β; a nucleic acid sequence encoding cytokines IP-10, SLC, MIP-1α, and MIP-1β; a nucleic acid sequence encoding cytokines IP-10, SLC, RANTES, MIP-1α, and MIP-1β; a nucleic acid sequence encoding cytokines IP-10, SLC, and ELC; a nucleic acid sequence encoding cytokines IP-10, SLC, ELC, and RANTES; a nucleic acid sequence encoding cytokines IP-10 , SLC, ELC, and MIP-1α; a nucleic acid sequence encoding cytokines IP-10, SLC, ELC, and MIP-1β; a nucleic acid sequence encoding cytokines IP-10, SLC, ELC, RANTES, and MIP-1α; a nucleic acid sequence encoding cytokines IP-10, SLC, ELC, RANTES, and MIP-1β; a nucleic acid sequence encoding cytokines IP-10, SLC, ELC, MIP-1α, and MIP-1β; and a nucleic acid sequence encoding cytokines IP-10, SLC, ELC, RANTES, MIP-1α, and MIP-1β.

In one embodiment, the virus vector is an expression vector. Methods for constructing a recombinant viral vector and controlling the expression of a polypeptide factor, such as utilizing tissue, cell, or developmental stage-specific promoter, enhancer, attenuator, terminator, etc, are well-known in the art. The expression of the encoded chemokines may be constitutively. In a preferred embodiment, the chemokine expression is temporally and/or spatially regulated, such as only expressing in a neoplastic cell. Controlled expression of the chemokine is advantageous, in that it may minimize toxicity or harmful side-effects in a subject to whom the composition is administered.

The recombinant virus, in particular, the vaccinia virus, composition disclosed herein may further comprise at least one nucleic acid sequence encoding at least one costimulatory factor. In one embodiment, the costimulatroy factor is a microorganism (e.g. bacteria or virus) antigen/pathogen or a neoplastic antigen/pathogen, which may be transported to the cell surface or secreted out of the cell after being expressed in a host cell. Methods of introducing a molecule to the cell surface or secreting a molecule out of the cell after/during synthesis, such as adding a nucleic acid sequence encoding a signal sequence in the N- or C-terminal of the costimulatory factor, are well established in the art.

In one embodiment, the costimulatory factor, such as a chemokine, is of animal origin. In a preferred embodiment, at least one costimulatory factor/chemokine encoded by the nucleic acid is a human or murine costimulatory factor/chemokine.

The composition of the present invention may be used to deliver at least one costimulatory factor, such as a chemokine, to a target cell. The target cell may be any cell of a mammal, including wild animals (e.g., primates, ungulates, rodents, felines, and canines), domestic animals (e.g., dog, cat, chicken, duck, goat, pig, cow, and sheep), and humans. In a preferred embodiment, the target cell is a human or murine cell. The delivery of the costimulatory factor may be performed in vitro, in vivo, in situ, or ex vivo. By way of example, the target cell is a neoplastic cell or a microorganism-infected cell (such as a HIV infected cell).

In one embodiment, the target cell which hosts the recombinant virus and expresses the costimulatory factor is amplified in vitro. The amplified host cell may be used as a research and development tool, for example, for the purpose of screening agonists, antagonists, inhibitors, and other modulators which may further contribute and/or facilitate the prevention and treatment of an infectious disease or a neoplasm. The host cell may also be reintroduced into a subject in order to facilitate the prevention and treatment of an infectious disease or a neoplasm. In another embodiment, the present invention provides a host animal which comprises the costimulatory factor-encoding vector composition and/or the host cell disclosed herein.

In one aspect, the present invention provides a pharmaceutical composition comprising the recombinant virus (preferably, vaccinia virus) composition and a pharmaceutically acceptable carrier. The pharmaceutically acceptable carrier must be “acceptable” in the sense of being compatible with the other ingredients of the composition, and not deleterious to the recipient thereof. The pharmaceutically acceptable carrier employed herein is selected from various organic or inorganic materials that are used as materials for pharmaceutical formulations, and which may be incorporated as analgesic agents, buffers, binders, disintegrants, diluents, emulsifiers, excipients, extenders, glidants, solubilizers, stabilizers, suspending agents, tonicity agents, vehicles, and viscosity-increasing agents. If necessary, pharmaceutical additives, such as antioxidants, aromatics, colorants, flavor-improving agents, preservatives, and sweeteners, may also be added. Examples of acceptable pharmaceutical carriers include carboxymethyl cellulose, crystalline cellulose, glycerin, gum arabic, lactose, magnesium stearate, methyl cellulose, powders, saline, sodium alginate, sucrose, starch, talc, and water, among others.

The composition of the present invention may be prepared by methods well-known in the pharmaceutical arts. For example, the composition may be brought into association with a carrier or diluent, as a suspension or solution. Optionally, one or more accessory ingredients (e.g., buffers, flavoring agents, surface active agents, and the like) also may be added. The choice of carrier will depend upon the route of administration of the composition. Formulations of the composition may be conveniently presented in unit dosage, or in such dosage forms as aerosols, capsules, elixirs, emulsions, eye drops, injections, liquid drugs, pills, powders, granules, suppositories, suspensions, syrup, tablets, or troches, which can be administered orally, topically, or by injection, including, but not limited to, intravenous, intraperitoneal, subcutaneous, intramuscular, and intratumoral (i.e. direct injection into the tumor) injection.

The composition of the present invention may be useful for administering an costimulatory factor or other anti-infection or anti-neoplasm agent to a subject to treat a variety of infectious disorders and neoplasm. As used herein, the “subject” is a mammal, including, without limitation, a cow, dog, human, monkey, mouse, pig, or rat. Preferably, the subject is a human.

The pharmaceutical composition is provided in an amount effective to treat the disorder in a subject to whom the composition is administered. As used herein, the phrase “effective to treat the disorder” means effective to ameliorate or minimize the clinical impairment or symptoms resulting from the infectious disease or neoplasia. For example, the clinical impairment or symptoms of the neoplasia may be ameliorated or minimized by diminishing any pain or discomfort suffered by the subject; by extending the survival of the subject beyond that which would otherwise be expected in the absence of such treatment; by inhibiting or preventing the development or spread of the neoplasia; or by limiting, suspending, terminating, or otherwise controlling the proliferation of cells in the neoplasm.

The amount of pharmaceutical composition that is effective to treat infectious diseases and neoplasia in a subject will vary depending on the particular factors of each case, including, for example, the type or stage of the infection or neoplasia, the subject's weight, the severity of the subject's condition, and the method of administration. These amounts can be readily determined by a skilled artisan. In general, the dosage of microorganism (within the therapeutic composition) to be administered to a subject may range from about 1 to 1×10⁹ pfu, preferably from about 1×10² to 5×10⁷ pfu, and, more preferably, from about 5×10² to 1×10⁷ pfu.

In the method of the present invention, the pharmaceutical composition may be administered to a human or animal subject by known procedures, including, without limitation, oral administration, parenteral administration (e.g., epifascial, intracapsular, intracutaneous, intradermal, intramuscular, intraorbital, intraperitoneal, intraspinal, intrasternal, intravascular, intravenous, parenchymatous, or subcutaneous administration), transdermal administration, and administration by osmotic pump. One preferred method of administration is parenteral administration, by intravenous or subcutaneous injection.

For oral administration, the formulation of the pharmaceutical composition may be presented as capsules, tablets, powders, granules, or as a suspension. The formulation may have conventional additives, such as lactose, mannitol, corn starch, or potato starch. The formulation also may be presented with binders, such as crystalline cellulose, cellulose derivatives, acacia, corn starch, or gelatins. Additionally, the formulation may be presented with disintegrators, such as corn starch, potato starch, or sodium carboxymethylcellulose. The formulation also may be presented with dibasic calcium phosphate anhydrous or sodium starch glycolate. Finally, the formulation may be presented with lubricants, such as talc or magnesium stearate.

For parenteral administration, the pharmaceutical composition may be combined with a sterile aqueous solution, which is preferably isotonic with the blood of the subject. Such a formulation may be prepared by dissolving a solid active ingredient in water containing physiologically-compatible substances, such as sodium chloride, glycine, and the like, and having a buffered pH compatible with physiological conditions, so as to produce an aqueous solution, then rendering said solution sterile. The formulation may be presented in unit or multi-dose containers, such as sealed ampules or vials. The formulation also may be delivered by any mode of injection, including any of those described above. Where an infection or a neoplasm is localized to a particular portion of the body of the subject, it may be desirable to introduce the pharmaceutical composition directly to that area by injection or by some other means (e.g., by intra-tumoral delivery, local delivery, or introducing the therapeutic composition into the blood or another body fluid).

For transdermal administration, the pharmaceutical composition may be combined with skin penetration enhancers, such as propylene glycol, polyethylene glycol, isopropanol, ethanol, oleic acid, N-methylpyrrolidone, and the like, which increase the permeability of the skin to the therapeutic composition, and permit the pharmaceutical composition to penetrate through the skin and into the bloodstream. The pharmaceutical composition also may be further combined with a polymeric substance, such as ethylcellulose, hydroxypropyl cellulose, ethylene/vinylacetate, polyvinyl pyrrolidone, and the like, to provide the composition in gel form, which may be dissolved in solvent, such as methylene chloride, evaporated to the desired viscosity, and then applied to backing material to provide a patch. The pharmaceutical composition may be administered transdermally, at or near the site on the subject where the neoplasm is localized. Alternatively, the pharmaceutical composition may be administered transdermally at a site other than the affected area, in order to achieve systemic administration.

The pharmaceutical composition of the present invention also may be released or delivered from an osmotic mini-pump or other time-release device. The release rate from an elementary osmotic mini-pump may be modulated with a microporous, fast-response gel disposed in the release orifice. An osmotic mini-pump would be useful for controlling release, or targeting delivery, of the pharmaceutical composition.

In accordance with the method of the present invention, the pharmaceutical composition may be administered to a subject who has infection or neoplasia, either alone or in combination with one or more antibiotics or antineoplastic drugs. Examples of antibiotics with which the pharmaceutical composition may be combined include, without limitation, penicillin, tetracycline, bacitracin, erythromycin, cephalosporin, streptomycin, vancomycin, D-cycloserine, fosfomycin, cefazolin, cephaloglycin, cephalexin, amphotericin B, gentamicin, tobramycin, kanamycin, and variants and derivatives thereof. Examples of antineoplastic drugs with which the pharmaceutical composition may be combined include, without limitation, carboplatin, cyclophosphamide, doxorubicin, etoposide, and vincristine. Additionally, when administered to a subject, the pharmaceutical composition may be combined with other anti-infection or anti-neoplastic therapies, including, without limitation, surgical therapies, radiotherapies, gene therapies, and immunotherapies.

In one aspect, the present invention provides a method for treating or preventing a neoplasm or infectious disease in a subject, comprising administering to the subject a pharmaceutical composition comprising a recombinant virus vector which comprises a nucleic acid sequence encoding at least one costimulatory factor. In one embodiment, the recombinant virus is a recombinant pox virus. Preferably, the recombinant pox virus is a vaccinia virus. More preferably, the recombinant pox virus is the WR strain of vaccinia virus.

In one embodiment, the present invention provides a method for treating or preventing a neoplasm or infectious disease in a subject, comprising administering to the subject a pharmaceutical composition comprising a recombinant vaccinia virus, wherein the virus comprises a nucleic acid sequence selected from the group consisting of: a nucleic acid sequence encoding cytokines IP-10 and ELC; a nucleic acid sequence encoding chemokines IP-10, ELC, and RANTES; a nucleic acid sequence encoding chemokines IP-10, ELC, and MIP-1α; a nucleic acid sequence encoding chemokines IP-10, ELC, and MIP-1β; a nucleic acid sequence encoding chemokines IP-10, ELC, RANTES, and MIP-1α; a nucleic acid sequence encoding chemokines IP-10, ELC, RANTES, and MIP-1β; a nucleic acid sequence encoding chemokines IP-10, ELC, MIP-1α, and MIP-1β; a nucleic acid sequence encoding chemokines IP-10, ELC, RANTES, MIP-1α, and MIP-1β; a nucleic acid sequence encoding chemokines IP-10 and SLC; a nucleic acid sequence encoding chemokines IP-10, SLC, and RANTES; a nucleic acid sequence encoding chemokines IP-10, SLC, and MIP-1α; a nucleic acid sequence encoding chemokines IP-10, SLC, and MIP-1β; a nucleic acid sequence encoding chemokines IP-10, SLC, RANTES, and MIP-1α; a nucleic acid sequence encoding chemokines IP-10, SLC, RANTES, and MIP-1β; a nucleic acid sequence encoding chemokines IP-10, SLC, MIP-1α, and MIP-1β; a nucleic acid sequence encoding chemokines IP-10, SLC, RANTES, MIP-1α, and MIP-1β; a nucleic acid sequence encoding chemokines IP-10, SLC, and ELC; a nucleic acid sequence encoding chemokines IP-10, SLC, ELC, and RANTES; a nucleic acid sequence encoding chemokines IP-10, SLC, ELC, and MIP-1α; a nucleic acid sequence encoding chemokines IP-10, SLC, ELC, and MIP-1β; a nucleic acid sequence encoding chemokines IP-10, SLC, ELC, RANTES, and MIP-1α; a nucleic acid sequence encoding chemokines IP-10, SLC, ELC, RANTES, and MIP-1β; a nucleic acid sequence encoding chemokines IP-10, SLC, ELC, MIP-1α, and MIP-1β; and a nucleic acid sequence encoding chemokines IP-10, SLC, ELC, RANTES, MIP-1α, and MIP-1β. In a preferred embodiment, the recombinant vaccinia virus is an expression vector. In another embodiment, the pharmaceutical composition used in the method to prevent and treat infectious diseases and neoplasms comprises a pharmaceutically acceptable carrier.

The recombinant virus, in particular, the vaccinia virus, composition used in a method to prevent and treat infectious diseases and neoplasms disclosed herein may further comprise at least one nucleic acid sequence encoding at least one costimulatory factor. In one embodiment, the costimulatory factor is a microorganism (e.g. bacteria or virus) antigen/pathogen or a neoplastic antigen/pathogen. In a preferred embodiment, the microorganism or neoplastic antigen/pathogen, after being expressed in a host cell, is relocated to cell surface or secreted out of the cell. Methods of introducing a molecule to cell surface or secreting a molecule out of the cell after/during synthesis are well established in the art.

In one embodiment, the pharmaceutical of the present invention further comprises at least one anti-neoplasm or anti-infection agent. As used herein, the term “agent” shall include any protein, polypeptide, peptide, nucleic acid (including DNA, RNA, and genes), antibody, Fab fragment, molecule, compound, antibiotic, drug, and any combinations thereof. The agent of the present invention may have any activity, function, or purpose. By way of example, the agent may be a diagnostic agent, a labeling agent, a preventive agent, or a therapeutic or pharmacologic agent.

As used herein, a “diagnostic agent” is an agent that is used to detect a disease, disorder, or illness, or is used to determine the cause thereof. As further used herein, a “labeling agent” is an agent that is linked to, or incorporated into, a cell or molecule, to facilitate or enable the detection or observation of that cell or molecule. By way of example, the labeling agent of the present invention may be an imaging agent or detectable marker, and may include any of those chemiluminescent and radioactive labels known in the art. The labeling agent of the present invention may be, for example, a nonradioactive or fluorescent marker, such as biotin, fluorescein (FITC), acridine, cholesterol, or carboxy-X-rhodamine (ROX), which can be detected using fluorescence and other imaging techniques readily known in the art. Alternatively, the labeling agent may be a radioactive marker, including, for example, a radioisotope, such as a low-radiation isotope. The radioisotope may be any isotope that emits detectable radiation, and may include ³⁵S, ³²P, ³H, radioiodide (¹²⁵I- or ¹³¹I-), or ⁹⁹mTc-pertechnetate (⁹⁹mTcO₄). Radioactivity emitted by a radioisotope can be detected by techniques well known in the art. For example, gamma emission from the radioisotope may be detected using gamma imaging techniques, particularly scintigraphic imaging.

Additionally, as used herein, the term “preventive agent” refers to an agent, such as a prophylactic, that helps to prevent a disease, disorder, or illness in a subject. As further used herein, the term “therapeutic” refers to an agent that is useful in treating a disease, disorder, or illness (e.g., a neoplasm) in a subject. In one embodiment, the anti-neoplasm or anti-infection agent used in a method to prevent and treat infectious diseases and neoplasms is an antibody. In a preferred embodiment, the antibody is preferably a mammalian antibody (e.g., a human antibody) or a chimeric antibody (e.g., a humanized antibody). More preferably, the antibody is a human or humanized antibody. As used herein, the term “humanized antibody” refers to a genetically-engineered antibody in which the minimum portion of an animal antibody (e.g., an antibody of a mouse, rat, pig, goat, or chicken) that is generally essential for its specific functions is “fused” onto a human antibody. In general, a humanized antibody is 1-25%, preferably 5-10%, animal; the remainder is human. Humanized antibodies usually initiate minimal or no response in the human immune system. Methods for expressing fully human or humanized antibodies in organisms other than human are well known in the art (see, e.g., U.S. Pat. No. 6,150,584, Human antibodies derived from immunized xenomice; U.S. Pat. No. 6,162,963, Generation of xenogenetic antibodies; and U.S. Pat. No. 6,479,284, Humanized antibody and uses thereof). In one embodiment of the present invention, the antibody is a single-chain antibody. In a preferred embodiment, the single-chain antibody is a human or humanized single-chain antibody. In another preferred embodiment of the present invention, the antibody is a murine antibody.

In one embodiment of the present invention, the anti-neoplasm or anti-infection agent may be a nucleic acid (e.g., plasmid) encodes or comprises at least one gene-silencing cassette, wherein the cassette is capable of silencing the expression of genes that are essential or important for the survival or proliferation of the pathogens or neoplastic cell. It is well understood in the art that a gene may be silenced at a number of stages, including, without limitation, pre-transcription silencing, transcription silencing, translation silencing, post-transcription silencing, and post-translation silencing. In one embodiment of the present invention, the gene-silencing cassette encodes or comprises a post-transcription gene-silencing composition, such as antisense RNA or RNAi. Both antisense RNA and RNAi may be produced in vitro, in vivo, ex vivo, or in situ.

For example, the anti-neoplasm or anti-infection agent of the present invention may be an antisense RNA. Antisense RNA is an RNA molecule with a sequence complementary to a specific RNA transcript, or mRNA, whose binding prevents further processing of the transcript or translation of the mRNA. Antisense molecules may be generated, synthetically or recombinantly, with a nucleic-acid vector expressing an antisense gene-silencing cassette. Such antisense molecules may be single-stranded RNAs or DNAs, with lengths as short as 15-20 bases or as long as a sequence complementary to the entire mRNA. RNA molecules are sensitive to nucleases. To afford protection against nuclease digestion, an antisense deoxyoligonucleotide may be synthesized as a phosphorothioate, in which one of the nonbridging oxygens surrounding the phosphate group of the deoxynucleotide is replaced with a sulfur atom (Stein et al., Oligodeoxynucleotides as inhibitors of gene expression: a review. Cancer Res., 48:2659-68, 1998).

Antisense molecules designed to bind to the entire mRNA may be made by inserting cDNA into an expression plasmid in the opposite or antisense orientation. Antisense molecules may also function by preventing translation initiation factors from binding near the 5′ cap site of the mRNA, or by interfering with interaction of the mRNA and ribosomes (e.g., U.S. Pat. No. 6,448,080, Antisense modulation of WRN expression; U.S. patent application No. 2003/0018993, Methods of gene silencing using inverted repeat sequences; U.S. patent application No., 2003/0017549, Methods and compositions for expressing polynucleotides specifically in smooth muscle cells in vivo; Tavian et al., Stable expression of antisense urokinase mRNA inhibits the proliferation and invasion of human hepatocellular carcinoma cells. Cancer Gene Ther., 10: 112-20, 2003; Maxwell and Rivera, Proline oxidase induces apoptosis in tumor cells and its expression is absent or reduced in renal carcinoma. J. Biol. Chem., e-publication ahead of print, 2003; Ghosh et al., Role of superoxide dismutase in survival of Leishmania within the macrophage. Biochem. J., 369:447-52, 2003; and Zhang et al., An anti-sense construct of full-length ATM cDNA imposes a radiosensitive phenotype on normal cells. Oncogene, 17:811-8, 1998).

Oligonucleotides antisense to a member of the infection/neoplasm-related signal-transduction pathways/systems may be designed based on the nucleotide sequence of the member of interest. For example, a partial sequence of the nucleotide sequence of interest (generally, 15-20 base pairs), or a variation sequence thereof, may be selected for the design of an antisense oligonucleotide. This portion of the nucleotide sequence may be within the 5′ domain. A nucleotide sequence complementary to the selected partial sequence of the gene of interest, or the selected variation sequence, then may be chemically synthesized using one of a variety of techniques known to those skilled in the art, including, without limitation, automated synthesis of oligonucleotides having sequences which correspond to a partial sequence of the nucleotide sequence of interest, or a variation sequence thereof, using commercially-available oligonucleotide synthesizers, such as the Applied Biosystems Model 392 DNA/RNA synthesizer.

Once the desired antisense oligonucleotide has been prepared, its ability to prevent or treat infection or neoplasm then may be assayed. For example, the antisense oligonucleotide may be administered to a subject, such as a mouse or a human, and its effects on the disease may be determined using standard clinical and/or molecular biology techniques, such as Western-blot analysis and immunostaining.

It is within the confines of the present invention that oligonucleotides antisense to a member of the infection/neoplasm-related signal-transduction pathways/systems may be linked to another agent, such as a anti-infection or anti-neoplastic drug. Moreover, antisense oligonucleotides may be prepared using modified bases (e.g., a phosphorothioate), as discussed above, to make the oligonucleotides more stable and better able to withstand degradation.

The anti-infection or anti-neoplasm agent of the present invention also may be an interfering RNA, or RNAi, including small interfering RNA (siRNA). As used herein, “RNAi” refers to a double-stranded RNA (dsRNA) duplex of any length, with or without single-strand overhangs, wherein at least one strand, putatively the antisense strand, is homologous to the target mRNA to be degraded. As further used herein, a “double-stranded RNA” molecule includes any RNA molecule, fragment, or segment containing two strands forming an RNA duplex, notwithstanding the presence of single-stranded overhangs of unpaired nucleotides. Additionally, as used herein, a double-stranded RNA molecule includes single-stranded RNA molecules forming functional stem-loop structures, such that they thereby form the structural equivalent of an RNA duplex with single-strand overhangs. The double-stranded RNA molecule of the present invention may be very large, comprising thousands of nucleotides; preferably, however, it is small, in the range of 21-25 nucleotides. In a preferred embodiment, the RNAi of the present invention comprises a double-stranded RNA duplex of at least 19 nucleotides.

In one embodiment of the present invention, RNAi is produced in vivo by an expression vector containing a gene-silencing cassette coding for RNAi (see, e.g., U.S. Pat. No. 6,278,039, C. elegans deletion mutants; U.S. patent application No. 2002/0006664, Arrayed transfection method and uses related thereto; WO 99/32619, Genetic inhibition by double-stranded RNA; WO 01/29058, RNA interference pathway genes as tools for targeted genetic interference; WO 01/68836, Methods and compositions for RNA interference; and WO 01/96584, Materials and methods for the control of nematodes). In another embodiment of the present invention, RNAi is produced in vitro, synthetically or recombinantly, and transferred into the microorganism using standard molecular-biology techniques. Methods of making and transferring RNAi are well known in the art (see, e.g., Ashrafi et al., Genome-wide RNAi analysis of Caenorhabditis elegans fat regulatory genes. Nature, 421:268-72, 2003; Cottrell et al., Silence of the strands: RNA interference in eukaryotic pathogens. Trends Microbiol., 11:37-43, 2003; Nikolaev et al., Parc. A Cytoplasmic Anchor for p53. Cell, 112:29-40, 2003; Wilda et al., Killing of leukemic cells with a BCR/ABL fusion gene RNA interference (RNAi). Oncogene, 21:5716-24, 2002; Escobar et al., RNAi-mediated oncogene silencing confers resistance to crown gall tumorigenesis. Proc. Natl. Acad. Sci. USA, 98:13437-42, 2001; and Billy et al., Specific interference with gene expression induced by long, double-stranded RNA in mouse embryonal teratocarcinoma cell lines. Proc. Natl. Acad. Sci. USA, 98:14428-33, 2001).

The inventors further discovered that an SLC agent directly promotes the proliferation of CD4 T cell. In one embodiment, the present invention provides a method for treating or preventing a neoplasm or infectious disease in a subject, comprising administering to the subject a pharmaceutical composition comprising an SLC agent in an amount effective to promote the proliferation of CD4 T cells directly. As used herein, an SLC agent refers to an SLC polypeptide, a nucleic acid encoding an SLC polypeptide, and a compound or factor which mimics SLC's effects on CD4 T cells. The term “directly” denotes that the SLC agent administered (if the SLC is a nucleic acid, its polypeptide product) promotes the proliferation of CD4 T cells through directly interaction with the cell. For example, an SLC agent (e.g., an SLC polypeptide) may bind to a receptor on the surface of a CD4 T cell to promote its proliferation. An SLC agent may also translocate into a CD4 T cell to activate upstream or downstream components of SLC signal transduction pathway to promote cell proliferation. In a preferred embodiment, the SLC agent is an SLC polypeptide, or fragment, variant, or derivative thereof. In another preferred embodiment, the SLC agent comprises a nucleic acid sequence encoding an SLC polypeptide. The SLC encoding nucleic acid may be an expression vector. In a preferred embodiment, the expression vector is a recombinant vaccinia virus vector.

In one embodiment of the present invention, the method of the present invention further comprises administering to the subject a costimulatory factor. In a preferred embodiment, the costimulatory factor is selected from a group consisting of chemokines, cytokines, and T cell activation agents. The T cell activation agent may be any agent which is capable of activating T cell, including antibodies. In one embodiment, the T cell activation agent is an anti-CD3 antibody.

The SLC-comprising pharmaceutical composition used herein may further comprise at least one anti-neoplasm or anti-infection agent. The co-administration of an SLC agent and an anti-neoplasm/anti-infection agent may have synergistic effects in treating or preventing the disorder. In one embodiment, the anti-neoplasm or anti-infection agent is an antibody. In a preferred embodiment, the antibody is a human or humanized antibody. The pharmaceutical composition may further comprise a pharmaceutically acceptable carrier.

In one embodiment, the present invention further provides a method for promoting the proliferation of a CD4 T cell, comprising administering to the cell an SLC agent in an amount effective to promote the proliferation of the cell directly. In a preferred embodiment, the SLC agent is an SLC polypeptide, or fragment, variant, or derivative thereof. In another preferred embodiment, the SLC agent comprises a nucleic acid sequence encoding an SLC polypeptide. The SLC encoding nucleic acid may be an expression vector. In a preferred embodiment, the expression vector is a recombinant vaccinia virus vector. A costimulatory factor, such as a chemokine, cytokine, or T cell activation agent may be co-administered to enhance or facilitate the proliferation of the T cell. In one embodiment, the T cell activation agent is an antibody or its antigen-binding fragment. In a preferred embodiment, the antibody is a human or humanized antibody.

The present invention also provides a method for treating or preventing a neoplasm or infectious disease in a subject, comprising the steps of: obtaining or generating a culture of T cells; optionally, contacting the T cells with an amount of T cell activation agent effective to activate the T cells; contacting the T cells with an SLC agent effective to promote CD4 T cell proliferation; and introducing the proliferated T cells into the subject in an amount effective to treat the neoplasm or infectious disease. In a preferred embodiment, the SLC agent is an SLC polypeptide, or fragment, variant, or derivative thereof. In another preferred embodiment, the SLC agent comprises a nucleic acid sequence encoding an SLC polypeptide. The SLC encoding nucleic acid may be an expression vector. In a preferred embodiment, the expression vector is a recombinant vaccinia virus vector. A costimulatory factor, such as a chemokine, cytokine, or T cell activation agent may be co-administered to enhance or facilitate the proliferation of the T cell. In one embodiment, the T cell activation agent is an antibody. In a preferred embodiment, the antibody is a human or humanized antibody. In one embodiment, the method for treating or preventing a neoplasm or infectious disease in a subject may further comprise at least one anti-neoplasm or anti-infection agent. The co-administration of the CD4 T cells and an anti-neoplasm/anti-infection agent may have synergistic effects in treating or preventing the disorder. In one embodiment, the anti-neoplasm or anti-infection agent is an antibody. In a preferred embodiment, the antibody is a human or humanized antibody.

The present invention is described in the following Examples, which are set forth to aid in the understanding of the invention, and should not be construed to limit in any way the scope of the invention as defined in the claims which follow thereafter.

### EXAMPLES

### Example 1

### Animals

Six to eight week old female BALB/c mice were purchased from Charles River Laboratories (Wilmington, Mass.) and housed in pathogen free conditions at the Institute for Comparative Medicine of Columbia University according to approved institutional protocols.

### Example 2

### Cell Lines and Viruses

All cell lines were obtained from ATCC (Rockville, Md.) unless otherwise noted. BSC-1 cells and CV-1 cells are derived from African green monkey kidney cells, HeLa cells are derived from human cervical carcinoma cells, and 143B TK cells are derived from a human sarcoma cell line and lack the thymidine kinase (tk) gene. The BALB/c (H-2^{d}) derived mouse tumor cell line CT-26 is an undifferentiated colorectal adenocarcinoma (Brattain et al., Establishment of mouse colonic carcinoma cell lines with different metastatic properties. Cancer Res. 40:2142-6, 1980) that was transfected with the human carcinoembryonic antigen (CEA) gene (designated CT26-CEA), and was obtained from Dr. Jeffrey Schlom (National Cancer Institute, Bethesda, Md.). All cell lines were grown in DMEM containing 10% FCS, lOmM L-glutamine, 100 U/ml streptomycin and 100 U/ml penicillin (complete media, reagents from Gibco BRL, Grand Island, N.Y.).

The 2.43 and GK 1.5 (ATCC) hybridomas were cultured in complete media and Iscove's modified DMEM containing 1.5 g/L sodium bicarbonate, 4 mM L-glutamine and 20% FBS, respectively.

Wild type vaccinia virus (strain WR) was obtained from ATCC. All viruses were grown to high titers in HeLa cells, and purified over sucrose gradients as described elsewhere (Broder and Earl. Design and construction of recombinant vaccinia viruses. Methods Mol. Biol. 62:173-97, 1997).

### Example 3

### Recombinant Vaccinia Virus Construction

The construction of recombinant vaccinia viruses has been described previously (id.) and was applied with slight modifications. Briefly, mSLC was amplified by PCR from a plasmid provided by Dr. Martin Dorf (University of California, Berkeley, Calif.) using the following primers flanking the gene, with additional nucleotides for KpnI and SalI restriction sites: F-AGACGTCGACCTCAAACTCAACCACAATC and R-ATTACGGTACCTCCAGGCG GGCTACTGGG, and cloned into the KpnI and SalI sites of the recombinant vaccinia pSC65 plasmid (a generous gift from Dr. Bernard Moss, NIH, Bethesda, Md.) under control of the synthetic vaccinia early/late promoter. The plasmid also contains the selectable marker LacZ under the control of the vaccinia P_{7.5} promoter. The pSC65 plasmid containing the SLC gene was transfected into wild type vaccinia infected CV1 cells using lipofectamine (Gibco BRL) according to standard protocols. An empty pSC65 plasmid was similarly transfected to construct the recombinant vaccinia virus expressing only LacZ (rVLacZ) as a negative control. Infected cells were collected and thymidine kinase deleted virus was selected by infecting 143B TK cells in the presence of 5-bromodeoxyuridine (BrdU, Sigma, St. Louis, Mo.). Cells from wells with single plaques were assumed to have developed from a single virus. Several such wells were individually collected, used to infect BSC-1 cells for 24 hours, and overlaid with agarose containing 2×DMEM supplemented with 5% heat-inactivated FCS, 2% LMP-agarose (Gibco BRL) and 5-bromo-4-chloro-3-indolyl-β-D-galactosidase (X-gal, Sigma). Infection was allowed to continue until blue plaques were clearly visualized. Several plaque isolates were selected and individually infected on BSC-1 cells, plaques with recombinant virus were selected and grown to high titers in HeLa cells. All viruses used in experiments were purified over a sucrose gradient as described and titers were determined on BSC-1 cells using a standard viral plaque assay (id.).

### Example 4

### Southern Blot Analysis

BSC-1 cells were infected with rVmSLC or rVLacZ control virus at an MOI of 10. Infected cells were maintained in DMEM containing 2.5% FCS for 48 hours, collected and lysed. DNA was extracted with phenol:chloroform and concentrated in ethanol using standard protocols. DNA was separated on a 2% agarose gel and transferred to a nitrocellulose membrane. The mSLC gene was detected using a DNA probe for SLC and the location of the gene in the thymidine kinase region was confirmed with a DNA probe for TK. Membranes were visualized using digoxigenin detection kit (Roche Molecular Biochemicals, Mannheim, Germany).

### Example 5

### Western Blot Analysis

BSC-1 cells were infected with rVmSLC or rVLacZ control virus at an MOI of 10. Infected cells were maintained in DMEM containing 2.5% FCS for 48 hours, collected and lysed. Proteins were resolved on a 15% SDS-PAGE gel and transferred to a nitrocellulose membrane (Bio-Rad, Hercules, Calif.). Recombinant murine SLC protein (R&D Systems, Minneapolis, Minn.) was used as a positive control. The membranes were washed and incubated with anti-mSLC goat polyclonal IgG (R&D Systems) at a dilution of 1:100. Blots were developed using biotin labeled anti-goat IgG mAb (R&D Systems) at a dilution of 1:10,000 and enhanced chemiluminescence detection reagents (Amersham-Pharmacia Biotech, Arlington Heights, Ill.) following manufacturer's instructions.

### Example 6

### Microchemotaxis Assay

Functional activity of secreted SLC protein was measured by migration across a 5 μm polycarbonate membrane (Costar, Cambridge, Mass.) in a microchemotaxis assay. BSC-1 cells were infected with either rVmSLC or rVLacZ (MOI=10) in DMEM containing 0.5% FCS. After 48 hours, supernatants from infected cells were collected. Supernatants or recombinant mSLC protein control (1 μg/ml), was used either directly in a chemotaxis assay or incubated for 60 minutes with anti-mSLC polyclonal Ab (5 μg/ml, Santa Cruz Biotech, Santa Cruz, Calif.) to specifically neutralize the activity of mSLC. Enriched T cells were derived from BALB/c spleens by passage over nylon wool columns. T cells were added to the upper chamber and migration was allowed to occur for three hours at 37° C. Cells in the lower chamber of the transwell were collected, and counted using Trypan blue exclusion in a blinded fashion. Three replicate wells were used for each culture condition and data represents one of three individual experiments. For flow cytometric analysis of chemotactic cells, cells in the lower chamber of the transwell were collected and 50,000 15 μm unlabeled polystyrene beads (Bangs Laboratories, Fishers, Ind.) were added. Flow cytometry proceeded by counting 5,000 bead events. The number of cells was determined with the following formula: (# of counted cells/5000)×50,000). Migration index was determined by dividing the number of cells migrating in a given treatment by the number of cells migrating in response to conditioned medium.

### Example 7

### ELISA

BSC-1 cells were infected with either rVmSLC or rVLacZ (MOI=10) in DMEM containing 0.5% FCS. After 48 hours, supernatants from infected cells were collected. Concentrations of SLC in the supernatants were determined using the Opteia Mouse TCA4 ELISA set (Pharmingen, San Diego, Calif.) according to manufacturers instructions.

### Example 8

### Cytotoxicity Assay

Effector cells were prepared from murine splenocytes after the indicated time and treatment. Single cell suspensions were prepared followed by lysis of red blood cells (RBCs) using ACK lysing buffer. Anti-vaccinia CTL were evaluated using vaccinia infected, or uninfected CT26-CEA cells as targets. Target cells were labeled with 100 μCi Na-chromate⁵¹ (⁵¹Cr, Amersham-Pharmacia Biotech) and used as targets in a standard 4 hour ⁵¹Cr-release assay. For anti-tumor CTL activity, effector cells were processed in the same way at the indicated time points, and uninfected CT26-CEA or parental CT26 cells, were used as targets. Cells were plated at various E:T ratios in triplcates in U-bottom 96 well plates. The ⁵¹Cr release in cell culture supernatants was measured using a Wallac Microbeta Tri-lux scintillation counter (PE Biosystems, Boston, Mass.) and the percentage specific release was calculated by the following formula: % Specific Lysis=[(Experimental release-Spontaneous release)/(Maximum release-Spontaneous Release)]×100.

### Example 9

### Establishment and Treatment of Subcutaneous Tumors

Tumors were established by s.c. injection of 5×10⁵ CT26-CEA cells into the shaved right flank of Balb/c mice. Ten mice were included in the treatment arms and five mice in the PBS control arm. On day 5 after tumor challenge, when palpable tumors were between 5 and 7 mm in diameter, tumors were injected with rVmSLC or rVLacZ (×10⁷ pfu) or PBS. For tumor treatment experiments, tumors were reinjected with virus or PBS on day 9 after tumor challenge. Tumors were evaluated by caliper every 1-3 days by measuring two perpendicular diameters and the area was determined by multiplying the two diameters. Survival was also monitored and mice were followed until tumors reached 100 mm² for two successive measurements. For tumor weight, tumors were removed intact at the indicated time points and weighed. These experiments were repeated three times.

### Example 10

### Immunohistochemical Analysis of Tumors

Established tumors were injected with rVmSLC or rVLacZ as described above and removed five days after treatment, fixed in IHC zinc fixative (BD Pharmingen) for 36 hours, embedded in paraffin and processed into 5 μm sections for immunohistochemical staining. Paraffin was removed from the sections in three changes of xylene and the sections were then rehydrated. Non-specific peroxidase activity was blocked in 1% hydrogen peroxide and non-specific proteins were blocked in 0.1 mg/ml BSA. Sections were then incubated with a monoclonal anti-mouse CD3 Ab developed for immunohistochemistry (clone 145-2C11, BD Pharmingen) for 24 hours at 4° C. Staining was visualized using ABC reagents (Vector Laboratories, Burlingame, Calif.) and DAB according to manufacturer's instructions. Sections were counterstained with hematoxylin and mounted with permount (Vector Labs).

### Example 11

### Flow Cytometric Analysis of Tumors

To detect cellular infiltrates in tumor tissue at various time points after tumor treatment, the subcutaneous tumors were removed, individually weighed, and digested in 1 mg/ml collagenase IV (Sigma) for 1 hour at 37° C. EDTA (10 mM) was added to the suspension and tumors were allowed to continue digestion for an additional 15-20 minutes. Tumor suspensions were washed, filtered through a 50 μm cell strainer and counted by Trypan blue exclusion. Samples were labeled with the following phycoerythrin (PE)-labeled antibodies: CD4 (L3T4) and CD8 (Ly-2). Antibodies were obtained from BD Pharmingen and used at dilutions of 1:100. The number of infiltrating cells was determined by FACSCalibur and analyzed using Cell Quest Software (BD Pharmingen) and normalized to the tumor weight by the following formula: (% positive cells×total cells)/(weight of tumor (g)).

### Example 12

### Depletion

For in vivo depletion of CD4 and CD8 T cells, ascites was generated by injection of pristine-primed nude mice with the GK1.5 and 2.43 hybridomas, respectively. 100 μl of ascites containing anti-CD4 or anti-CD8, or the combination of both was given i.p. on days −3, −2, −1, 0, 5, 10, and every 7 days thereafter (relative to tumor implantation). Depletion was monitored by flow cytometry of splenocytes once per week beginning on day −1 in age-matched littermates.

### Example 13

### Proliferation Assay

RBC-depleted splenocytes were either used directly or enriched for T cells using a pan T cell isolation kit (Miltenyi Biotech, Auburn, Calif.) using the manufacturer's protocol, and assessed for purity by flow cytometry for CD3 expression (>95%). Cells were cultured in triplicate in a 96-well plate in the presence or absence of increasing concentrations of SLC protein. Plates were incubated for 72 hours, with ³H-Thymidine (Amersham Pharmacia) added for the final 12 hours. Cells were collected by cell harvester, and thymidine incorporation was measured using a Wallac Microbeta Tri-lux scintillation counter (PE Biosystems).

### Example 14

### Statistical Analysis

Statistical differences between treatment groups were determined by Student's t-test, while tumor growth was analyzed by two-way ANOVA using Bonferroni post-tests. All analysis was performed using GraphPad Prism software and P values below 0.05 were considered significant.

### Example 15

### Construction of a Recombinant Vaccinia Virus Expressing the SLC Gene

The inventors amplified murine SLC DNA from a plasmid containing the full-length cDNA sequence. The cloned segment included a 500 bp DNA fragment encoding SLC, flanked by KpnI and SalI restriction sites. This fragment was inserted into the KpnI/SalI site of the recombinant vaccinia plasmid, pSC65, which places the SLC gene under a vaccinia early/late promoter. The plasmid also contains LacZ (a selectable marker) and segments of the vaccinia thymidine kinase (TK) gene allowing homologous recombination into the non-essential TK region of vaccinia virus. The insert was sequenced to ascertain both the presence of the gene and to verify that there were no mutations of the inserted sequence (data not shown). The plasmid was used for homologous recombination into wild type vaccinia virus as described in Materials and Methods. Insertion into the TK region of the vaccinia virus was confirmed by both PCR and Southern blot analysis of vaccinia infected cells (data not shown). Similarly, the empty pSC65 plasmid was used to construct the control virus, rVLacZ.

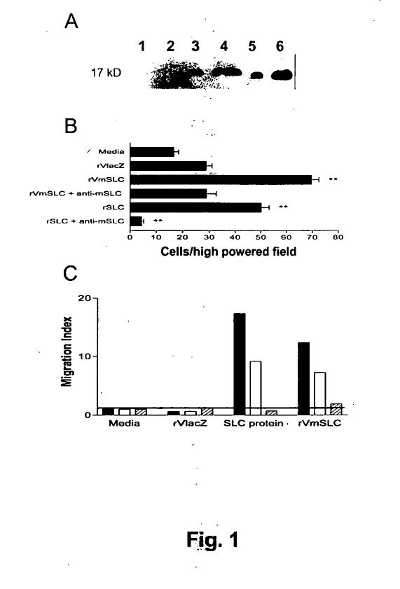

The expression of SLC protein was analyzed by infecting BSC-1 cells with rVmSLC or rVLacZ and SLC protein was detected in lysates of infected cells by Western blot. A polyclonal anti-mSLC antibody recognized a 14 kDa protein within lysates of rVmSLC-infected cells that was absent from rVLacZ-infected cells (FIG. 1A). This band corresponded to the band observed with recombinant mSLC protein control. Thus, cells infected with rVmSLC produced SLC protein in vitro.

### Example 16

### Infection of Cells with RVMSLC Results in the Secretion of Biologically Active SLC Protein

The functional activity of SLC released from rVmSLC-infected cells was tested in an in vitro chemotaxis assay. Naïve T cells showed over a two-fold increase in chemotactic activity mediated by the cell culture supernatant of rVmSLC-infected cells compared to the rVLacZ supernatant (FIG. 1B). Flow cytometry of the migrating cells confirmed a similar pattern of migration with a significant increase in the migration of T cells (FIG. 1C). Interestingly, there was an especially strong induction of CD4 T cell migration.

The increased chemotaxis induced by rVmSLC was specifically due to the presence of SLC as the addition of neutralizing anti-mSLC antibody abrogated the chemotactic effect (FIG. 1B). The slight migration observed with the antibody treated rVmSLC supernatant was due to the high concentration of SLC secreted by rVmSLC-infected cells, as determined by ELISA (data not shown). Thus, SLC is secreted from rVmSLC-infected cells and induces the migration of T cells and DCs in vitro, with a particularly powerful effect on CD4 T cells.

### Example 17

### Mice Tolerate Infections of RVMSLC up to 1×10⁷ PFU

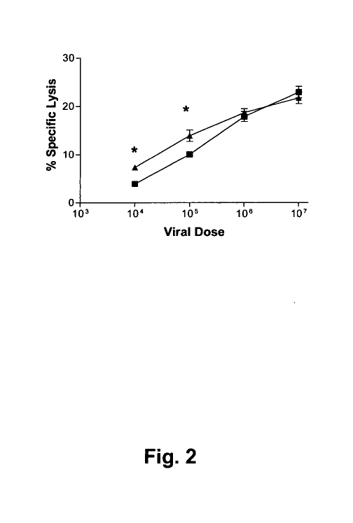

Pox viruses possess a variety of genes aimed at altering the host immune system to escape detection, including chemokine binding proteins and chemokine mimics, suggesting the possibility that expression of SLC could influence viral pathogenicity or the host immune response to viral challenge (Murphy, P. M., Viral exploitation and subversion of the immune system through chemokine mimicry. Nat. Immunol. 2:116-22, 2001). To evaluate the virulence of rVmSLC, mice were injected i.p. with between 1×10⁴ and 1×10⁷ pfu of either rVmSLC or rVLacZ, and were observed for toxicity. There were no differences in appearance of the mice after vaccination with rVmSLC compared to rVLacZ at any dose tested. Immunogenicity was evaluated by determining vaccinia-specific cytotoxic T cell responses by standard ⁵¹Cr release assay. Although there was no significant difference in vaccinia-specific CTL induced by rVmSLC or rVLacZ at high doses of virus administration (10⁶-10⁷ pfu), mice receiving a dose of 1×10⁵ pfu or below of rVmSLC demonstrated minimally enhanced anti-vaccinia CTL responses when rVmSLC was used compared to rVLacZ (FIG. 2A). Thus, cell-mediated immunity, though slightly enhanced when animals received lower doses of virus remained largely unaffected by the secretion of mSLC.

### Example 18

### Intratumoral Injection of RVMSLC Promotes the Infiltration of CD4 T Cells into the Tumor

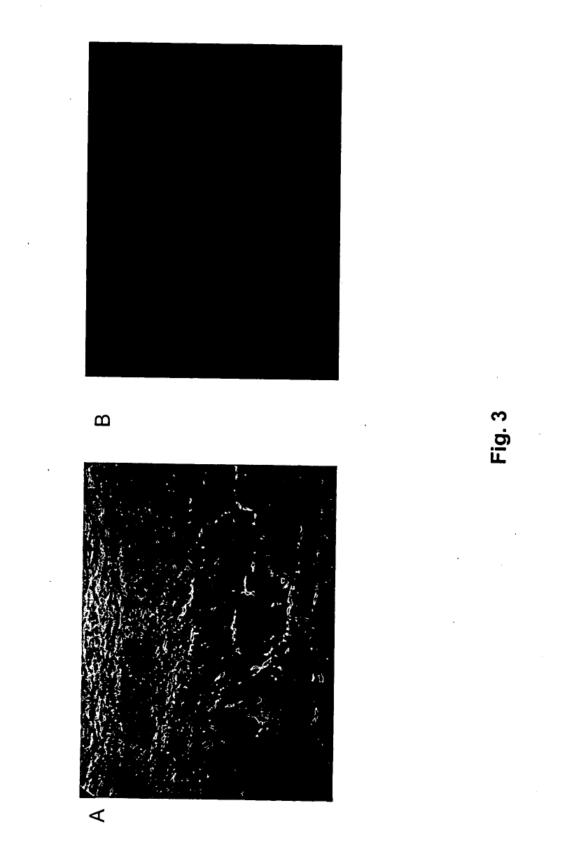

SLC is chemotactic for T cells both in vitro and in vivo (Gunn et al., A chemokine expressed in lymphoid high endothelial venules promotes the adhesion and chemotaxis of naive T lymphocytes. Proc. Natl. Acad. Sci. USA 95:258-63, 1998; Chan et al., Secondary lymphoid-tissue chemokine (SLC) is chemotactic for mature dendritic cells. Blood 93:3610-6, 1999). The inventors therefore tested whether intratumoral injection of rVmSLC could induce the infiltration of T cells and DCs into injected tumors. To detect the presence of T cells, the tumors were collected five days after injection with rVmSLC or rVLacZ, fixed and stained with an anti-CD3 mAb for immunohistochemical staining. Both vaccines induced focal areas of T cell infiltration, presumably at the site of virus injection underscoring the adjuvant properties of vaccinia virus (FIG. 3). However, rVmSLC injected tumors (FIG. 3A) contained a higher infiltration of T cells than rVLacZ injected tumors (FIG. 3B).

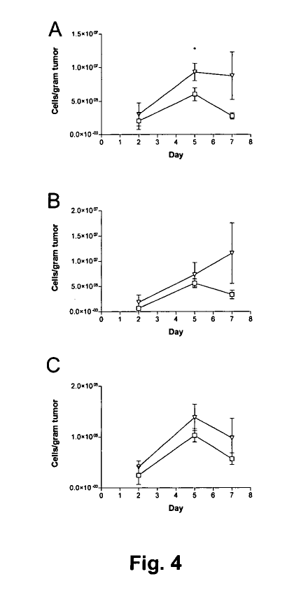

To better quantitate the degree of T cell and DC infiltration into the tumor, the inventors generated single cell suspensions of individual vaccinia treated tumors, stained them with PE-labeled antibodies and determined the kinetics of cellular infiltration by collecting tumor samples at different times after virus injection. Although 2 days after treatment, there was little difference between the infiltrates in either group, by day 5 there was a significant increase in the number of CD4T cells per gram of tissue within the rVmSLC treated tumors compared to the rVLacZ control group (FIG. 4A). On day 7 there was also an increase in the number of CD4 T cells per gram of tumor tissue in the rVmSLC injected tumors compared to rVLacZ treated tumors, although this was not statistically significant (p=0.07). Although there was a difference in the number of CD8 T cells in tumors compared to rVLacZ treated tumors until day 7, this also failed to reach significance (FIG. 4B. p=0.11). Infiltration of tumors with DCs (FIG. 4C) increased to a maximum on day 5 after injection, but there was no discernible difference in DC numbers between the treatment groups.

### Example 19

### Intratumoral Injection of RVMSLC Inhibits Tumor Growth

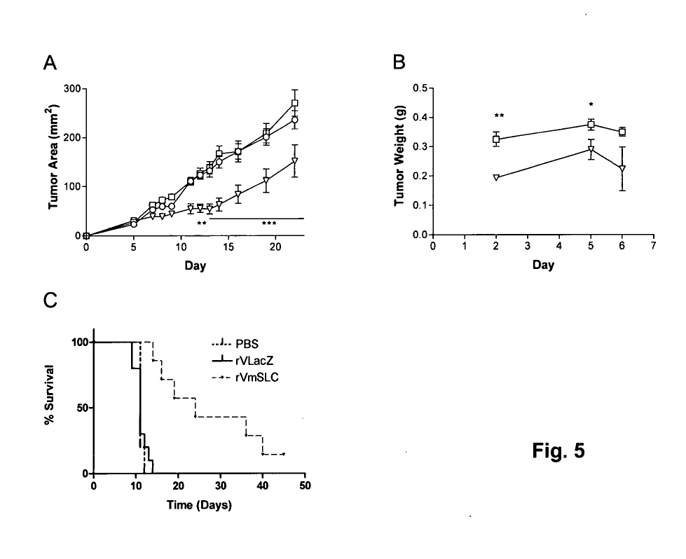

In order to determine if intratumoral injection of rVmSLC had a therapeutic effect on established subcutaneous tumors, Balb/c mice were injected s.c. with 5×10⁵ CT26-CEA tumor cells and treated with 1×10⁷ pfu of either rVmSLC or rVLacZ or PBS, on days 5 and 9 after tumor implantation. While tumors treated with rVLacZ grew at virtually the same rate as tumors treated with PBS, the growth rate of rVmSLC treated tumors was significantly decreased (p<0.01, FIG. 5A). Tumor weight was also decreased in rVmSLC treated mice at all time points evaluated up to 7 days after tumor injection (FIG. 5B). The data shown is representative of three individual experiments with similar results. Thus, local injection of rVmSLC into established tumors significantly inhibited tumor growth and this did not appear to be due to non-specific tumor cell lysis by vaccinia virus since rVLacZ had no effect at the same dose in this model.

### Example 20

### The Antitumor Response of RVMSLC is Mediated by CD4 T Cells

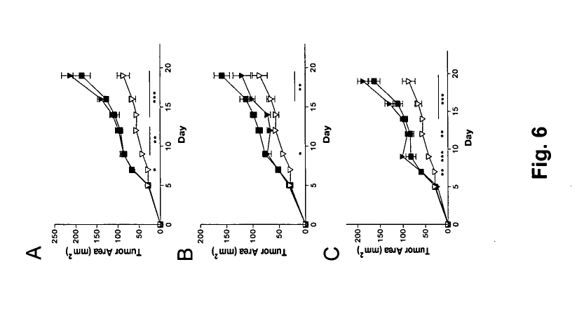

Previous studies with other chemokines have implicated CD8 T cells in the rejection of CT26 tumor cells (Ruehlmann et al., MIG (CXCL9) chemokine gene therapy combines with antibody-cytokine fusion protein to suppress growth and dissemination of murine colon carcinoma. Cancer Res. 61:8498-503, 2001). Therefore, CD8 T cell responses were evaluated by chromium release assay and no differences in tumor specific CTL activity were detected (data not shown). The mechanism of the anti-tumor response observed with rVmSLC was further evaluated by depleting mice of CD4 T cells, CD8 T cells or both. While mice depleted of CD8 T cells (FIG. 6B) exhibited some delay in tumor growth after treatment with rVmSLC, depletion of CD4 T cells (FIG. 6A) completely abrogated the anti-tumor effects of rVmSLC. As expected, depletion of both CD4 and CD8 T cells completely inhibited the effects of rVmSLC on tumor growth (FIG. 6C). Thus, the therapeutic anti-tumor response observed after treatment with rVmSLC was mediated predominantly by CD4 T cells.

### Example 21

### SLC Induces the Proliferation of Lymphocytes In Vitro

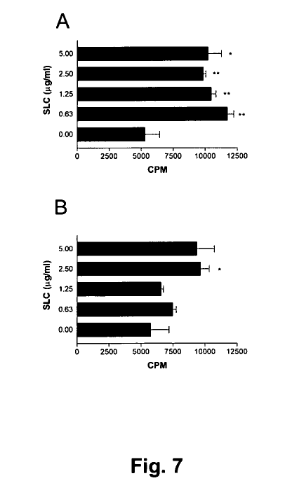

The observation that rVmSLC induced migration of CD4 T cells and that the anti-tumor effects were dependent on CD4 T cells suggests that SLC may be capable of regulating lymphocyte proliferation (Luther and Cyster, Chemokines as regulators of T cell differentiation. Nat. Immunol. 2:102-7, 2001). SLC protein was added in increasing doses to whole splenocytes (FIG. 7A) or enriched T cells (FIG. 7B) stimulated with suboptimal doses of anti-CD3, and proliferation was determined by [³H]-thymidine incorporation. Murine SLC induced proliferation of activated splenocytes as well as enriched T cells. These results demonstrate that mSLC may have a direct effect on T-cell proliferation following antigen recognition, in addition to the well characterized chemotactic functions of SLC.

Discussed below are results obtained by the inventors in connection with the experiments of Examples 1-21:

In this report the inventors demonstrated that functional murine SLC could be expressed in vaccinia virus and recombinant rVmSLC induced migration of T-cells in vitro and in vivo. The rVmSLC was also able to mediate regression of five day established subcutaneous tumors in mice suggesting a significant therapeutic effect was possible after local delivery. Regressing tumors were infiltrated by DCs and CD8 T cells, but the most pronounced infiltration was by CD4 T cells following vaccine administration. Although others have reported enhanced DC infiltration in systems using SLC to treat tumors, no differences were seen in the number of infiltrating DCs in rVmSLC treated tumors versus rVLacZ treated tumors (Kirk et al., The dynamics of the T-cell antitumor response: chemokine-secreting dendritic cells can prime tumor-reactive T cells extranodally. Cancer Res. 61:8794-802, 2001). This may be due to the direct lytic effect of replication competent vaccinia virus, which induces DC apoptosis and cell death, even though the virus also stimulates pro-inflammatory signals attracting DCs. Nonetheless, the data on rVmSLC is consistent with previous reports demonstrating regression of established tumors after local injection of recombinant SLC protein or an HSV amplicon expressing SLC (Moss, B., Vaccinia virus: a tool for research and vaccine development. Science 252:1662-7, 1991; Kirk et al., supra; Sharma et al., Secondary lymphoid organ chemokine reduces pulmonary tumor burden in spontaneous murine bronchoalveolar cell carcinoma. Cancer Res. 61:6406-12, 2001). Thus, SLC appears to be able to mediate local anti-tumor responses, which occurs through the attraction of both mature DCs and naive T cells, key mediators of anti-tumor immunity. Although the induction of tumor-specific immune responses is generally thought to occur predominantly in secondary lymphoid tissue, the use of peripheral chemokine expression at sites of tumor growth represents a method for priming T cells in the periphery (Ochsenbein et al., Roles of tumour localization, second signals and cross priming in cytotoxic T-cell induction. Nature 411:1058-64, 2001).

In addition to localizing the cells involved in priming tumor-specific immune responses, effective tumor immunity also requires activation, or co-stimulation, of T cells since most TAAs are weak and/or self antigens. The use of vaccinia virus as a delivery vector offers several advantages, most notably the strong adjuvant properties of the virus which provide local inflammatory responses and additional signals for activating adaptive immunity. Vaccinia virus also provides a stable vector for chemokine expression and has been used to deliver immune modulatory genes to established tumors both in mice and in cancer patients (Kaufman et al., A phase I trial of intra lesional RV-B7.1 vaccine in the treatment of malignant melanoma. Hum. Gene Ther. 11: 1065-82, 2000; Kaufman et al., A phase I trial of intralesional rV-Tricom vaccine in the treatment of malignant melanoma. Hum. Gene Ther. 12:1459-80, 2001). However, the expression of functional chemokines by pox viruses is not straightforward, since most pox viruses, including vaccinia, encode a variety of genes that subvert the chemokine system in order to avoid immune detection (Murphy, supra). There are a number of viral chemokine antagonists that have been described, as well as chemokine receptor mimics, highlighting the importance of chemokines in pox virus-host interactions. Studies of deletional mutants have further suggested that alteration of the chemokine modulating genes can influence both viral pathogenicity and immunogenicity. For example, the myxoma virus encodes an IFNγ-R homolog, M-T7, which binds a variety of CC and CXC chemokines (Mossman et al., Myxoma virus M-T7, a secreted homolog of the interferon-gamma receptor, is a critical virulence factor for the development of myxomatosis in European rabbits. Virology 215:17-30, 1996). Rabbits infected with myxoma virus lacking M-T7 exhibited increased leukocyte infiltration at the site of infection. Similarly, a 35 kDa soluble vaccinia protein can bind and inhibit a spectrum of CC chemokines, including SLC (Alcami et al., Blockade of chemokine activity by a soluble chemokine binding protein from vaccinia virus. J Immunol. 160:624-33, 1998). The vaccinia WR (V-WR) strain was selected for vaccine development because it does not express this 35 kDa chemokine binding protein. Expression of mSLC did not appear to alter the pathogenicity of the virus, as all mice tolerated doses of up to 1×10⁷ pfu.

In order to determine if mSLC expression influenced viral immunogenicity, the inventors evaluated anti-vaccinia CTL responses following rVmSLC administration. While there were no differences in CTL activity at doses above 1×10⁶ pfu, there was a subtle, yet reproducible, increase in CTL responses after rVmSLC vaccination at doses of 1×10⁵ and below. This suggests that expression of mSLC may minimally enhance the immunogenicity of V-WR and it is possible that this effect may be more pronounced in attenuated vaccinia strains where large numbers of immune regulatory genes are deleted. The expression of chemokines and other immune stimulatory genes in vaccinia may provide an approach for administration of lower viral doses while maintaining adequate anti-vaccinia immunity. Thus, effective vaccination may be possible with lower doses of virus, which may reduce the adverse reactions observed at higher doses of vaccinia virus.

Chemokines are known to have pleiotropic functions including chemotaxis, angiogenic or angiostatic functions, and may directly influence effector cells. The anti-tumor effect observed in the experimental model was due to immune mediated effectors as depletion of T cells completely abrogated the effect of rVmSLC. Furthermore, the mechanism of tumor rejection was found to be dependent on CD4 T cells, in particular. The data supporting the role of CD4 T cells in this model includes the preferential migration of CD4 T cells in vitro (FIG. 1C), the accumulation of CD4 T cells in rVmSLC injected tumors (FIG. 4A) and the complete loss of therapeutic responses in CD4 T cell depleted mice (FIG. 6B). These results do not necessarily rule out the possibility that CD8 T cells also contribute to tumor rejection since small effects may have been obscured by the use of vaccinia virus, a potent activator of CD8 T cells (Titu et al., The role of CD8(+) T cells in immune responses to colorectal cancer. Cancer Immunol. Immunother. 51:235-47, 2002).

In addition to the migratory effects of SLC on CD4 T cells, the observation that recombinant SLC stimulates proliferation of T cells in vitro suggests that SLC may also act to directly activate selected T cells. This may explain the therapeutic effectiveness of SLC after local delivery and is consistent with previous studies demonstrating that the in vivo concentration of SLC may play a role in the expansion of CD4 T cells in the steady state to maintain a homeostatic population of CD4, but not CD8 T cells (Ploix et al., A ligand for the chemokine receptor CCR7 can influence the homeostatic proliferation of CD4 T cells and progression of autoimmunity. J. Immunol. 167:6724-30, 2001). In mice treated with an SLC antagonist, there was a decrease in the severity of graft-vs-host disease (GVHD) following adoptive transfer of allogeneic splenocytes (Sasaki et al., Antagonist of secondary lymphoid-tissue chemokine (CCR ligand 21) prevents the development of chronic graft-versus-host disease in mice. J. Immunol. 170:588-96, 2003). The amelioration of GVHD symptoms was due to a selective inhibition of CD4 T cells by the SLC antagonist. Thus, the anti-tumor effect induced by rVmSLC may be due to an increased number of T cells or to the direct activation and expansion of CD4 T cells at the tumor site, or both. The concept that chemokines can directly (co)stimulate T cells has been reported for CCL5 (RANTES), CCL3 and 4 (MIP-1α and 1β), and CCL2 (MCP-1), which have been shown to induce T cell proliferation and IL-2 production in the context of anti-CD3 activation (Wong and Fish, Chemokines: attractive mediators of the immune response. Semin. Immunol. 15:5-14, 2003; Romagnani, S., Cytokines and chemoattractants in allergic inflammation. Mol. Immunol. 38:881-5, 2002; Luther and Cyster, supra). The CCR5-ligands, CCL3, 4 and 5, exert a positive regulatory effect on T_{H}1 differentiation by inducing IL-12 or IFN-γ expression and by directly polarizing T_{H} cells (Wong and Fish, Chemokines: attractive mediators of the immune response. Semin. Immunol. 15:5-14, 2003; Romagnani, S., Cytokines and chemoattractants in allergic inflammation. Mol. Immunol. 38:881-5, 2002; Luther and Cyster, supra). The underlying mechanisms of chemokine function seem to involve signaling pathways, such as FAK activation and P13-kinase activation, and subsequent gene regulatory events that follow activation of cognate chemokine receptors (Luther and Cyster, supra; Dorner et al., MIP-1alpha, MIP-1beta, RANTES, and ATAC/lymphotactin function together with IFN-gamma as type 1 cytokines. Proc. Natl. Acad. Sci. USA 99:6181-6, 2002; Nanki and Lipsky, Stimulation of T-Cell activation by CXCL12/stromal cell derived factor-I involves a G-protein mediated signaling pathway. Cell Immunol. 214:145-54, 2001; Wong and Fish, supra). A distinct effect on T-cell proliferation has not been previously reported for SLC Thus, the normal physiological function of SLC in the lymph node environment may involve the colocalization of mature DCs and naive T cells, as well as the induction and amplification of CD4 T cells that come into contact with their cognate antigen.

Murine SLC can be efficiently expressed by vaccinia virus and local delivery to solid tumors resulted in effective therapeutic responses. The infiltration of rVmSLC injected tumors with T cells and DCs supports the notion that SLC is capable of establishing a cellular neolymphoid environment within the tumor, as suggested previously (Fan et al., Cutting edge: ectopic expression of the chemokine TCA4/SLC is sufficient to trigger lymphoid neogenesis. J. Immunol. 164:3955-9, 2000; Kirk et al., supra). The presence of live replicating vaccinia virus may provide an additional danger signal for supporting a local inflammatory response (Matzinger, supra; Kirk et al., supra). The benefit of a vaccinia vector for SLC delivery over protein injection may lie in the enhanced ability to draw immature DCs to the tumor site as a result of viral lysis of tumor cells. Because healthy animals likely have a low number of mature DCs, injection of rVmSLC might be a benefit over injection of chemokine protein due to an ability to draw both immature and mature DCs into the tumor. Thus, a likely scenario in the experimental model is that vaccinia virus promotes a pro-inflammatory environment conducive to the attraction of immature DCs to the site wherein they take up antigen including vaccinia infected and uninfected tumor cells. After DC maturation and CCR7 upregulation, the presence of local mSLC maintains an SLC gradient that “holds” the DCs within the tumors. The SLC gradient also acts to increase the number of naïve T cells migrating to the tumor site, thus enhancing the probability for T cell priming through direct contact of mature DCs with T cells. This may also explain why the inventors were unable to detect tumor specific CTLs in the spleens of vaccinated mice since effector T cells may be localized to the tumor mass at the time of the assay (data not shown) and the inventors plan to evaluate this possibility in the future. This result, however, is in agreement with Sharma et al, who demonstrated that local SLC treatment increased cytotoxic T cell activity against 3LL tumor cells only after exposure to re-stimulated lymph node-derived lymphocytes (Sharma, supra). In addition, local release of SLC may also directly activate CD4 T cells further enhancing anti-tumor immunity.

In summary, the data supports the use of pox viruses for the expression of chemokines and the use of such vectors for the local delivery of selected chemokines into established tumors. There have been a large number of pre-clinical and clinical trials documenting the induction of antigen specific T cell responses with a variety of vaccines. However, there has been less attention paid to the interaction of effector T cells and tumor cells at the site of established tumors. The emerging concept that progressing tumors induce an immunosuppressive environment implies that strategies altering the local microenvironment warrant investigation. Chemokines offer the potential to recruit highly specific cells to the tumor site and may also be able to influence the functional activity of recruited cell populations. Thus, the development of pox viruses expressing chemokines represents a compelling approach for the immunotherapy of solid tumors.

While the foregoing invention has been described in some detail for purposes of clarity and understanding, it will be appreciated by one skilled in the art, from a reading of the disclosure, that various changes in form and detail can be made without departing from the true scope of the invention in the appended claims.

1. A recombinant vaccinia virus composition comprising a nucleic acid sequence selected from the group consisting of:
(a) a nucleic acid sequence encoding chemokines IP-10 and ELC;
(b) a nucleic acid sequence encoding chemokines IP-10, ELC, and RANTES;
(c) a nucleic acid sequence encoding chemokines IP-10, ELC, and MIP-1α;
(d) a nucleic acid sequence encoding chemokines IP-10, ELC, and MIP-1β;
(e) a nucleic acid sequence encoding chemokines IP-10, ELC, RANTES, and MIP-1α;
(f) a nucleic acid sequence encoding chemokines IP-10, ELC, RANTES, and MIP-1β;
(g) a nucleic acid sequence encoding chemokines IP-10, ELC, MIP-1α, and MIP-1β;
(h) a nucleic acid sequence encoding chemokines IP-10, ELC, RANTES, MIP-1α, and MIP-1β;
(i) a nucleic acid sequence encoding chemokines IP-10 and SLC;
(g) a nucleic acid sequence encoding chemokines IP-10, SLC, and RANTES;
(k) a nucleic acid sequence encoding chemokines IP-10, SLC, and MIP-1α;
(l) a nucleic acid sequence encoding chemokines IP-10, SLC, and MIP-1β;
(m) a nucleic acid sequence encoding chemokines IP-10, SLC, RANTES, and MIP-α;
(n) a nucleic acid sequence encoding chemokines IP-10, SLC, RANTES, and MIP-1β;
(o) a nucleic acid sequence encoding chemokines IP-10, SLC, MIP-1α, and MIP-1β;
(p) a nucleic acid sequence encoding chemokines IP-10, SLC, RANTES, MIP-1α, and MIP-1β;
(r) a nucleic acid sequence encoding chemokines IP-10, SLC, and ELC;
(s) a nucleic acid sequence encoding chemokines IP-10, SLC, ELC, and RANTES;
(t) a nucleic acid sequence encoding chemokines IP-10, SLC, ELC, and MIP-1α;
(u) a nucleic acid sequence encoding chemokines IP-10, SLC, ELC, and MIP-1β;
(v) a nucleic acid sequence encoding chemokines IP-10, SLC, ELC, RANTES, and MIP-1α;
(w) a nucleic acid sequence encoding chemokines IP-10, SLC, ELC, RANTES, and MIP-1β;
(x) a nucleic acid sequence encoding chemokines IP-10, SLC, ELC, MIP-1α, and MIP-1β; and
(y) a nucleic acid sequence encoding chemokines IP-10, SLC, ELC, RANTES, MIP-1α, and MIP-1β.

2. The composition of claim 1, further comprising at least one nucleic acid sequence encoding at least one costimulatory factor.

3. The composition of claim 1, wherein at least one chemokine is derived from an animal source.

4. The composition of claim 3, wherein at least one chemokine is a human chemokine.

5. The composition of claim 3, wherein at least one chemokine is a murine chemokine.

6. The composition of claim 1, wherein the virus is an expression vector.

7. A host cell comprising the composition of claim 1.

8. An animal comprising the host cell of claim 7.

9. A pharmaceutical composition comprising the composition of claim 1 and a pharmaceutically acceptable carrier.

10. A method for treating or preventing a neoplasm or infectious disease in a subject, comprising administering to the subject a pharmaceutical composition comprising a recombinant vaccinia virus, wherein the virus comprises a nucleic acid sequence selected from the group consisting of:
(a) a nucleic acid sequence encoding chemokines IP-10 and ELC;
(b) a nucleic acid sequence encoding chemokines IP-10, ELC, and RANTES;
(c) a nucleic acid sequence encoding chemokines IP-10, ELC, and MIP-1α;
(d) a nucleic acid sequence encoding chemokines IP-10, ELC, and MIP-1β;
(e) a nucleic acid sequence encoding chemokines IP-10, ELC, RANTES, and MIP-1α;
(f) a nucleic acid sequence encoding chemokines IP-10, ELC, RANTES, and MIP-1β;
(g) a nucleic acid sequence encoding chemokines IP-10, ELC, MIP-1α, and MIP-1β;
(h) a nucleic acid sequence encoding chemokines IP-10, ELC, RANTES, MIP-1α, and MIP-1β;
(i) a nucleic acid sequence encoding chemokines IP-10 and SLC;
(g) a nucleic acid sequence encoding chemokines IP-10, SLC, and RANTES;
(k) a nucleic acid sequence encoding chemokines IP-10, SLC, and MIP-1α;
(l) a nucleic acid sequence encoding chemokines IP-10, SLC, and MIP-1β;
(m) a nucleic acid sequence encoding chemokines IP-10, SLC, RANTES, and MIP-1α;
(n) a nucleic acid sequence encoding chemokines IP-10, SLC, RANTES, and MIP-1β;
(o) a nucleic acid sequence encoding chemokines IP-10, SLC, MIP-1α, and MIP-1β;
(p) a nucleic acid sequence encoding chemokines IP-10, SLC, RANTES, MIP-1α, and MIP-1β;
(r) a nucleic acid sequence encoding chemokines IP-10, SLC, and ELC;
(s) a nucleic acid sequence encoding chemokines IP-10, SLC, ELC, and RANTES;
(t) a nucleic acid sequence encoding chemokines IP-10, SLC, ELC, and MIP-α;
(u) a nucleic acid sequence encoding chemokines IP-10, SLC, ELC, and MIP-1β;
(v) a nucleic acid sequence encoding chemokines IP-10, SLC, ELC, RANTES, and MIP-1α;
(w) a nucleic acid sequence encoding chemokines IP-10, SLC, ELC, RANTES, and MIP-1β;
(x) a nucleic acid sequence encoding chemokines IP-10, SLC, ELC, MIP-1α, and MIP-1β; and
(y) a nucleic acid sequence encoding chemokines IP-10, SLC, ELC, RANTES, MIP-1α, and MIP-1β.

11. The method of claim 10, further comprising at least one nucleic acid sequence encoding at least one costimulatory factor.

12. The method of claim 10, wherein the virus is an expression vector.

13. The method of claim 10, wherein the pharmaceutical composition further comprises a pharmaceutically acceptable carrier.

14. The method of claim 10, wherein the pharmaceutical composition further comprises at least one anti-neoplasm or anti-infection agent.

15. The method of claim 14, wherein the at least one anti-neoplasm or anti-infection agent is an antibody.

16. A method for treating or preventing a neoplasm or infectious disease in a subject, comprising administering to the subject a pharmaceutical composition comprising an SLC agent in an amount effective to directly promote the proliferation of CD4 T-cells.

17. The method of claim 16, further comprising administering to the subject a costimulatory factor.

18. The method of claim 17, wherein the costimulatory factor is selected from a group consisting of chemokines, cytokines, and T cell activation agents.

19. The method of claim 18, wherein the T cell activation agent is an antibody.

20. The method of claim 16, wherein the SLC agent is an SLC polypeptide, or fragment, variant, or derivative thereof.

21. The method of claim 16, wherein the SLC agent comprises a nucleic acid sequence encoding an SLC polypeptide.

22. The method of claim 21, wherein the nucleic acid is an expression vector.

23. The method of claim 21, wherein the nucleic acid is a vaccinia virus nucleic acid.

24. The method of claim 16, wherein the pharmaceutical composition further comprises at least one anti-neoplasm or anti-infection agent.

25. The method of claim 24, wherein the at least one anti-neoplasm or anti-infection agent is an antibody.

26. The method of claim 16, wherein the pharmaceutical composition further comprising a pharmaceutically acceptable carrier.

27. A method for promoting the proliferation of a CD4 T cell, comprising administering to the cell an SLC agent in an amount effective to directly promote the proliferation of the cell.

28. The method of claim 27, further comprising administering to the cell a costimulatory factor.

29. The method of claim 17, wherein the costimulatory factor is selected from a group consisting of chemokines, cytokines, and T cell activation agents.

30. The method of claim 29, wherein the T cell activation agent is an antibody.

31. The method of claim 27, wherein the SLC agent is an SLC polypeptide or fragment, variant, or derivative thereof.

32. The method of claim 27, wherein the SLC agent comprises a nucleic acid sequence encoding an SLC polypeptide.

33. The method of claim 32, wherein the nucleic acid is an expression vector.

34. The method of claim 32, wherein the nucleic acid is a vaccinia virus nucleic acid.

35. A method for treating or preventing a neoplasm or infectious disease in a subject, comprising the steps of:
(a) obtaining or generating a culture of T cells;
(b) optionally, contacting the T cells with an amount of T cell activation agent effective to activate the T cells;
(c) contacting the T cells with an SLC agent effective to directly promote CD4 T cell proliferation; and
(d) introducing the proliferated T cells into the subject in an amount effective to treat the neoplasm or infectious disease.

36. The method of claim 35, further comprising administering to the subject a costimulatory factor.

37. The method of claim 35, further comprising administering to the subject at least one anti-neoplasm or anti-infection agent.

38. The method of claim 37, wherein the at least one anti-neoplasm or anti-infection agent is an antibody.

39. The method of claim 35, wherein the SLC agent is an SLC polypeptide or fragment, variant, or derivative thereof.

40. The method of claim 35, wherein the SLC agent comprises a nucleic acid sequence encoding an SLC polypeptide.

41. The method of claim 40, wherein the nucleic acid is an expression vector.

42. The method of claim 40, wherein the nucleic acid is a vaccinia virus nucleic acid.


SEQUENCE LISTING (2 sequences)

SEQ ID NO: 1 - DNA, 29 bp, Artificial Sequence
  primer
agacgtcgacctcaaactcaaccacaatc

SEQ ID NO: 2 - DNA, 29 bp, Artificial Sequence
  Primer
attacggtacctccaggcgggctactggg